(19) United States
(12) Patent Application Publication
(10) Pub. No.: US 2011/0152915 A1
(43) Pub. Date: Jun. 23, 2011

(54) HEMOSTATIC STABILIZATION SYSTEM

(76) Inventor: Tamer Ibrahim, Pleasant Hill, CA (US)

(21) Appl. No.: 12/966,670

(22) Filed: Dec. 13, 2010

Related U.S. Application Data
(60) Provisional application No. 61/288,614, filed on Dec. 21, 2009.
(60) Provisional application No. 61/288,752, filed on Dec. 21, 2009.
(60) Provisional application No. 61/288,763, filed on Dec. 21, 2009.

(51) Int. Cl. A61M 29/00

(52) U.S. Cl. 606/194

(57) ABSTRACT

A hemostatic stabilization system for hemostatically accessing a bodily organ includes a hemostatic cup and a hemostatic port. The hemostatic cup includes a proximal end, a distal end, and a wall extending therebetween. The hemostatic cup also includes a tissue attachment edge at the distal end, and a sealing surface at the proximal end. The hemostatic port is configured for insertion through the sealing surface and into the bodily organ. The hemostatic port defines a working channel configured to receive an instrument to be inserted into the bodily organ. The hemostatic cup defines a vacuum chamber configured to adhere the hemostatic cup to the bodily organ when a vacuum source is coupled thereto and when the tissue attachment edge is brought into contact with the bodily organ. The hemostatic cup is configured to maintain a hemostatic environment when the instrument is inserted into the bodily organ through the hemostatic port.

### CROSS-REFERENCE TO RELATED APPLICATION

This application claims the benefit of U.S. Provisional Application No. 61/288,614 filed Dec. 21, 2009, the entirety of which is hereby incorporated by reference. This application is also related to U.S. Provisional Application Nos. 61/288,752 and 61/288,763, which are incorporated by reference in their entireties, herein.

### SUMMARY

In one embodiment, a hemostatic stabilization system facilitates delivery of instruments inside the heart or a vessel. In another embodiment, the hemostatic stabilization system provides a hemostatic working port for delivery of instruments inside the heart or to a vessel. For example, in one embodiment, the hemostatic stabilization system is used to deliver a mechanical or tissue valve, an annuloplasty ring, a valve annulus resizing element, an ablation catheter, or any other instrumentation useful for a variety of beating heart procedures. The hemostatic stabilization system may be used in conjunction with any surgical access technique, including sternotomy, thoracotomy, port access, etc.

In one embodiment, a hemostatic stabilization system includes a suction member. The suction member could be configured as a suction cup, or a suction halo, and has a central hemostatic working region. The suction member attaches to tissue and allows for incision into tissue while providing hemostatis by sealing around a centrally located port (or other working region or channel). The hemostatic port (or other working region or channel) may be used to introduce various instruments to the tissue, such as incisors or other therapeutic devices or instruments.

The central region of the hemostatic stabilization system can be configured as a double barrel to allow for delivery of a protected endoscope or other imaging probes adjacent to other surgical tools. The incision can be subsequently sealed with a bio glue, suture, patch, or other known closure device or technique.

### BRIEF DESCRIPTION OF THE DRAWINGS

FIG. 1 illustrates a hemostatic stabilization system in accordance with one embodiment;

FIG. 2 illustrates a cross-sectional side view of the hemostatic stabilization system of FIG. 1;

FIG. 3 illustrates a cross-sectional side view of another embodiment of a hemostatic stabilization device;

FIGS. 4A-4C illustrates embodiments of a hemostatic valve compatible with any of the embodiments of FIGS. 1-3;

FIG. 5 is a flow chart illustrating one method of port access into a body cavity suitable for use with a hemostatic stabilization system, including the embodiments of FIGS. 1-3;

FIG. 6 illustrates a cross-sectional side view of a hemostatic stabilization system in accordance with another embodiment; and

FIG. 7 is a flow chart illustrating another method of port access into a body cavity suitable for use with a hemostatic stabilization system, including the embodiment of FIG. 6.

### DETAILED DESCRIPTION

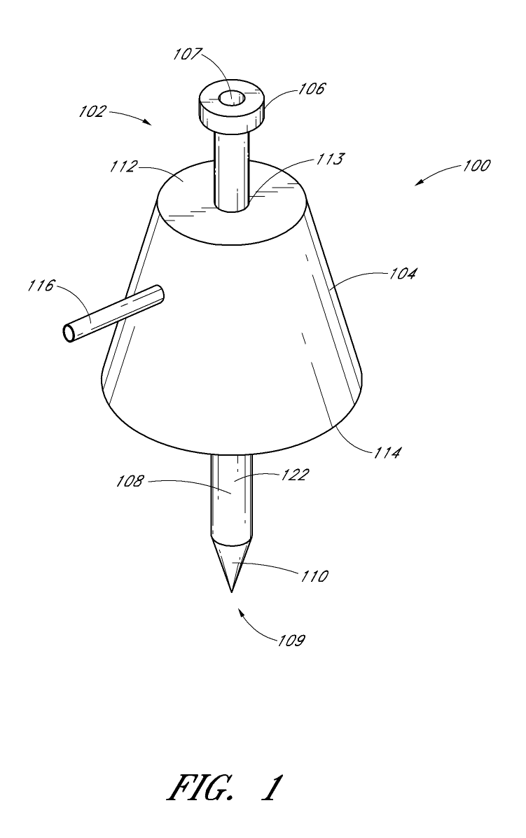

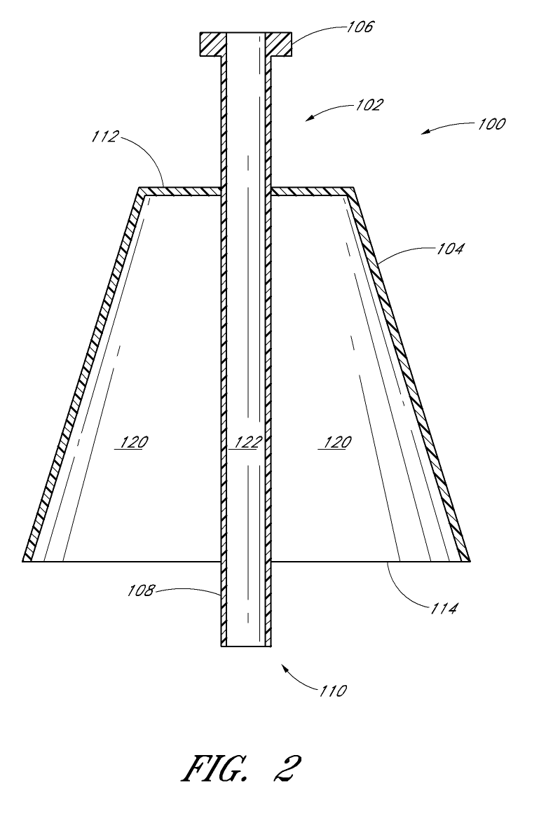

FIGS. 1 and 2 illustrate one embodiment of a hemostatic stabilization system 100 configured to provide hemostatic access to a bodily organ or cavity. The hemostatic stabilization system includes a hemostatic port 102 and a hemostatic cup 104. The hemostatic port 102 includes a hemostatic valve 106, cannula 108, and cannula tip 110. The hemostatic valve 106 includes a sealed proximal opening 107 and a distal opening 109. The sealed proximal opening 107 is configured to prevent liquids or gases within the cannula 108 to escape the hemostatic port 102. In addition, the proximal opening 107 is configured to allow instrument insertion into a bodily organ or cavity, as will be discussed in greater detail below.

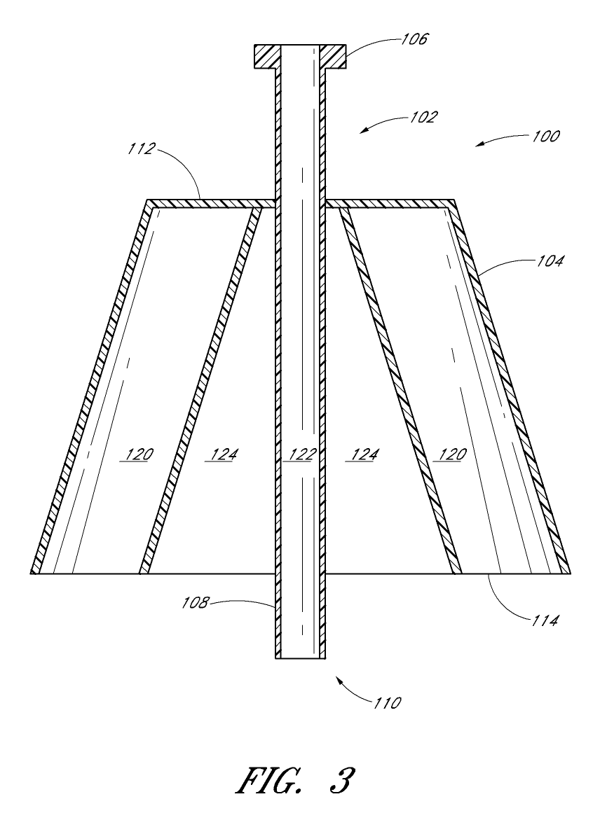

The hemostatic valve 106 is attached to the cannula 108 at the cannula's proximal end. The cannula 108 includes at least one lumen or working channel (for example, working channel 122, as shown in FIGS. 2 and 3), which provides communication between the hemostatic valve's 106 proximal opening 107 and the cannula's distal opening 109. The diameter of the working channel is sufficient for instrument insertion and manipulation to perform various surgical or other therapeutic treatments within a hemostatic environment within a medical patient. In some embodiments, the cannula 108 includes more than one lumen. For example, the cannula 108 may include two, three or more than three working channels. The cannula tip 110 can be formed as a point, as illustrated in FIG. 1, which can be sharp and suitable for piercing tissue for insertion of the cannula 108 therein. In other embodiments, the cannula tip 110 has a flat, rounded, or curved shaped. For example, the cannula tip 110 can have a blunt edge or an atraumatic edge at the distal opening 109.

The hemostatic cup 104 can have a frustoconical shape, as illustrated in FIG. 1. In other embodiments, the hemostatic cup 104 has a cylindrical or ovoid shape. The proximal end of the hemostatic cup 104 includes a sealing surface 112. The sealing surface 112 prevents fluids and gases contained within the hemostatic cup 104 from leaking or otherwise exiting the hemostatic stabilization system. The sealing surface 112 is configured to support and hold the hemostatic port 102 in place and to prevent leaks of fluids and gases at the interface 113 between the hemostatic port 102 and the sealing surface 112. In one embodiment, the sealing surface 112 is configured to allow the hemostatic port 102 to slide in and out of the hemostatic cup 104 while preventing leaks at the port-sealing surface interface.

The distal end of the hemostatic cup 104 defines a tissue attachment edge 114, which is shaped and sized to sealingly engage a tissue surface of a bodily organ or structure. For example, the tissue attachment edge 114 can be shaped as a circle having a diameter sufficient to surround the apex of a patient's heart. In one use, the tissue attachment edge 114 is placed into contact with the heart tissue surrounding the apex of a patient's heart. A vacuum source (e.g., vacuum pump, manual hand pump, etc.—not shown) is coupled to the hemostatic cup's vacuum port 116 to create a negative pressure within the hemostatic cup's vacuum chamber 120 (see FIGS. 2 and 3). The negative pressure within the vacuum chamber 120 causes the hemostatic cup 104 to adhere (e.g., via suction) to the patient's tissue. The hemostatic port 102 may then be advanced to contact and penetrate the tissue surrounded by the tissue attachment edge 114 of the hemostatic cup 104. The port 102 is advanced through the tissue and into the patient's cavity or organ (e.g., into the left ventricle of the patient's heart). Instruments may then be inserted through the proximal opening 107 of the port 102 and into the patient's cavity or organ, while maintaining a hemostatic working environment.

FIG. 2 shows additional detail of the hemostatic stabilization device 100. For example, FIG. 2 illustrates the vacuum chamber 120 formed within the hemostatic cup, and the working channel 122 for instrument insertion, formed within the hemostatic port 102. In addition, in the embodiment of FIG. 2, the hemostatic port 102 has an atraumatic cannula tip 110 at the distal end of the cannula 108.

FIG. 3 illustrates a cross-sectional view of another embodiment of a hemostatic stabilization device 100. From the outside, the hemostatic stabilization device 100 looks identical to the hemostatic stabilization device of FIG. 1. However, the hemostatic stabilization device 100 of FIG. 3 includes a vacuum chamber 120 and a port channel 124. The vacuum chamber 120 enables the hemostatic stabilization device 100 to be attached to a patient's tissue for hemostatic port access. The port channel 124 provides a separate, discrete channel for insertion and movement of the hemostatic port 102 relative to the hemostatic cup 104.

In some embodiments, the port channel 124 includes a vacuum port (not shown), similar to the vacuum port 116 shown in FIG. 1. The vacuum port can be attached to a vacuum source and used to create a negative pressure within the port channel 124. Providing a negative pressure in both of the vacuum chamber 120 and the port cannel 124, advantageously provides redundancy and a back-up in case one of the seals between the hemostatic cup 104 and the patient's tissue fails. In some embodiments, the hemostatic cup includes more than one vacuum chamber, such as two or three vacuum chambers. In addition, as discussed above, one of the vacuum chambers can also form the port channel of the hemostatic stabilization device 100.

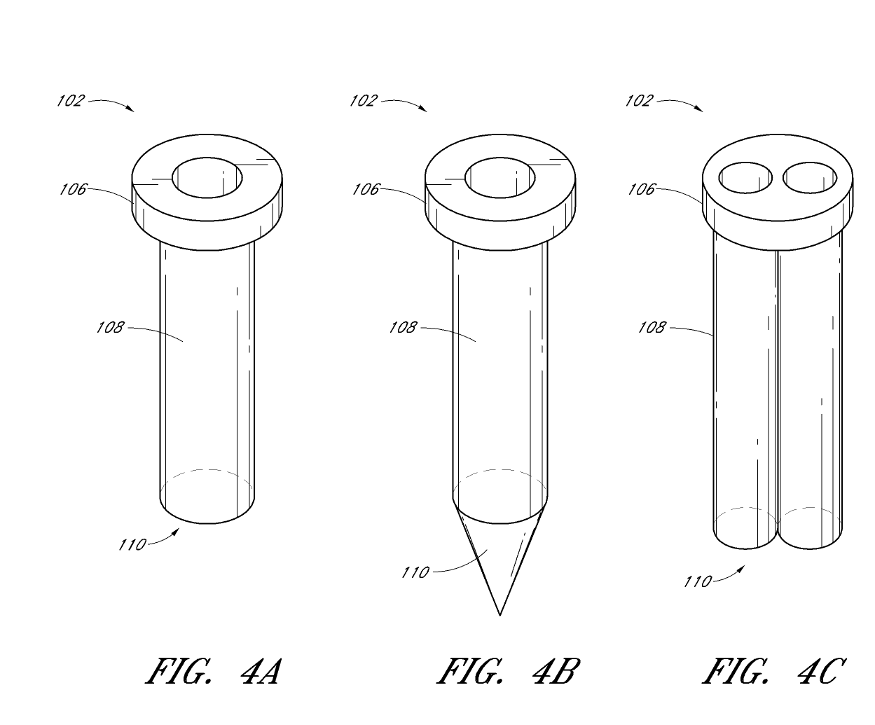

Additional embodiments of a hemostatic port 102 configured to be used with any of the hemostatic stabilization systems 100 discussed herein, are illustrated in FIGS. 4A-4C. The hemostatic port 102 of FIG. 4A has a single cannula 108 that has a cylindrical shape, a single working channel, and terminates in a flat, atraumatic tip 110. The hemostatic port 102 of FIG. 4B is similar to the port 102 of FIG. 4A, except that it terminates in an incising, self-dilating tip. The incising, self-dilating tip advantageously allows the hemostatic port 102 to be inserted into a hemostatic cup and advanced into and through target tissues without requiring additional instrumentation or surgical intervention. In some embodiments, the hemostatic port 102 is configured like a trocar and/or can include three channels.

Finally, the hemostatic port 102 of FIG. 4C includes a dual barrel configuration. The port 102 includes two cannulae 108, each defining its own working channel. The distal tips 110 of the cannulae 108 can each have a flat edge (as shown), an incising, self-dilating tip (as shown in FIG. 4b), or can be shaped to define a single incising, self-dilating surface. For example, each cannula 108 can be formed as half of an incising, self-dilating surface, such that when placed next to each other, the two cannulae 108 together form a single incising, self-dilating surface at their distal tips 110. In other embodiments, the hemostatic port 108 includes three or more then three cannulae 108.

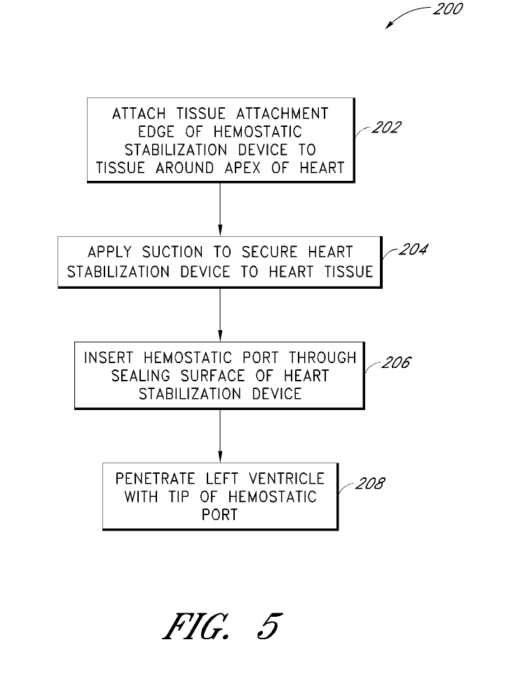

FIG. 5 illustrates a method of port access to a left ventricle. The method of FIG. 5 can be performed with any of the hemostatic stabilization systems described above. In addition, the method 200 of FIG. 5 may be performed to hemostatically access any bodily cavity or organ, in addition to the left ventricle. The method 200 begins at block 202, where the tissue attachment edge of a hemostatic stabilization device is attached to tissue around the apex of a patient's heart. At block 204, suction is applied to the hemostatic stabilization device to secure it to heart tissue. At block 206, a hemostatic port is inserted through the sealing surface of the hemostatic stabilization device. In some embodiments, the hemostatic port is already inserted through the sealing surface of the hemostatic stabilization device prior to application of suction, as performed at block 204. At block 208, the outside wall of the patient's left ventricle is penetrated with the tip of the hemostatic port. The hemostatic port may then be advanced into the patient's left ventricle. Instruments may then be inserted into the patient's left ventricle via the working channel of the hemostatic port. For example, in some embodiments, instruments and devices to replace a patient's native heart valve are inserted into the patient's left ventricle via the hemostatic port's working channel. Such instruments and devices include, but are not limited to, a dilation balloon, such as a balloon suitable for performing valvuloplasty, instruments to cut and remove the leaflets of a diseased native valve, and/or a replacement valve and instruments to attach the replacement valve to the patient's heart.

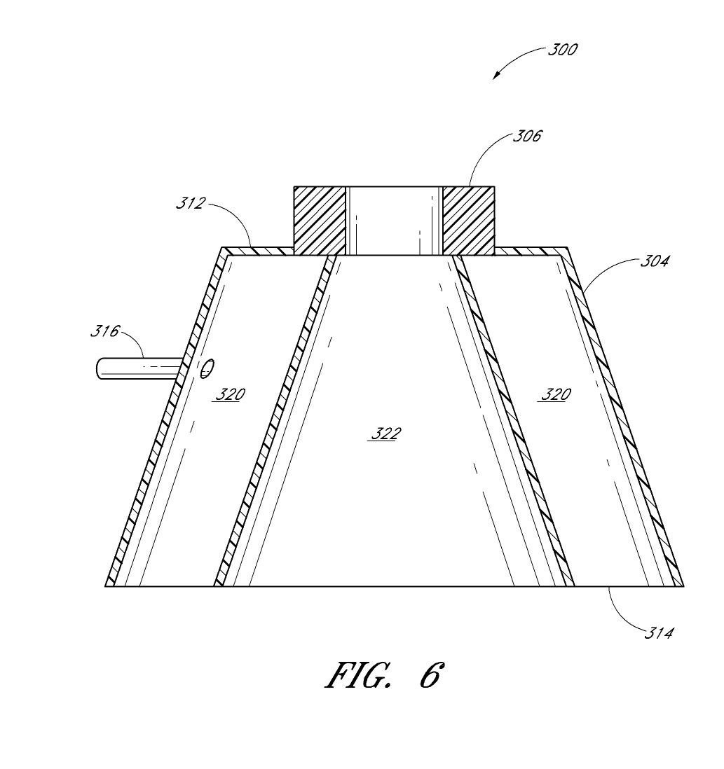

FIG. 6 illustrates another embodiment of a hemostatic stabilization system. The hemostatic stabilization system 300 is similar to the hemostatic stabilization system 100 of FIGS. 1-3, except that the hemostatic stabilization system 300 does not include a separate hemostatic port (e.g., hemostatic port 102). The hemostatic stabilization system 300 includes a hemostatic cup 304. A hemostatic valve 306 is provided at the hemostatic cup's proximal end. The proximal end is sealed with a sealing surface 312. The distal end defines a tissue attachment edge 314 for attachment to patient tissues. A vacuum chamber 320 communicates to a suction device (not shown) via a vacuum port 316. The suction device creates a pressure between the hemostatic cup and the patient's tissue, which causes the hemostatic cup 304 to attach to the patient's tissue. A working channel 322 within the hemostatic cup 304 provides a lumen or path for device and/or instrument insertion.

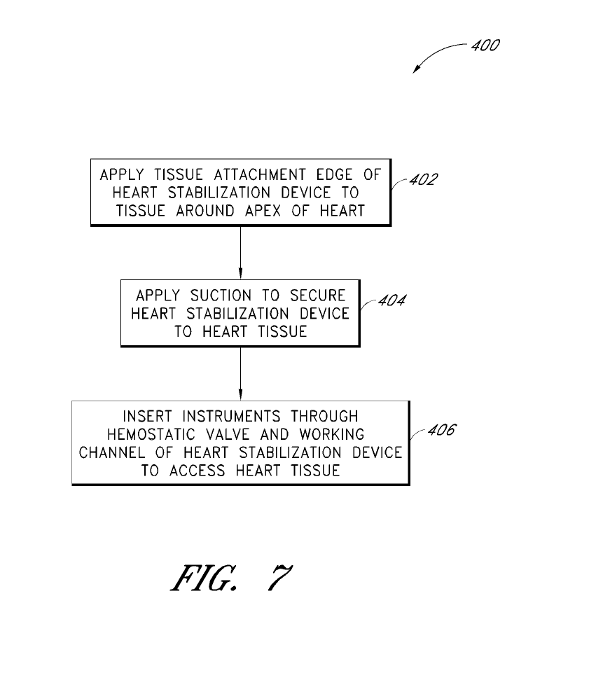

FIG. 7 illustrates a flow chart of another method of port access to a patient's left ventricle. The method 400 of FIG. 7 may be performed with the hemostatic stabilization system 300 of FIG. 6. In addition, the method 400 is not restricted to accessing a patient's left ventricle, but like the method 200 of FIG. 5, may be used to access any chamber of a patient's heart, any bodily cavity, or organ, as desired.

The method 400 begins at block 402. At block 402, the tissue attachment edge of a hemostatic stabilization system is attached to tissue around the apex of a patient's heart. At block 404, suction is applied to secure the hemostatic stabilization system to the patient's heart tissue. At block 406, instruments are inserted through the hemostatic valve and working channel of the hemostatic stabilization system to access heart tissue. The instruments may be used to incise an opening through the wall of the left ventricle at the apex so instruments and/or devices may be delivered to the heart through the left ventricle. For example, any of the procedures described above may be performed in conjunction with the method 400 of FIG. 7.

What is claimed is:

1. A hemostatic stabilization system for hemostatically accessing a bodily organ, comprising:
a hemostatic cup comprising a proximal end, a distal end, and a wall extending therebetween, the hemostatic cup further comprising a tissue attachment edge at the distal end, and a sealing surface at the proximal end; and
a hemostatic port configured for insertion through the sealing surface and into the bodily organ, the hemostatic port defining a working channel configured to receive an instrument to be inserted into the bodily organ,
wherein the hemostatic cup defines a vacuum chamber configured to adhere the hemostatic cup to the bodily organ when a vacuum source is coupled thereto and when the tissue attachment edge is brought into contact with the bodily organ, and
wherein the hemostatic cup is configured to maintain a hemostatic environment when the instrument is inserted into the bodily organ through the hemostatic port.

2. The hemostatic stabilization system of claim 1, wherein the hemostatic cup comprises a frustoconical shape.

3. The hemostatic stabilization system of claim 1, wherein the hemostatic cup further comprises a port channel through which the hemostatic port is provided.

4. The hemostatic stabilization system of claim 1, further comprising a second vacuum chamber.

5. The hemostatic stabilization system of claim 1, wherein the hemostatic port comprises an incising, self-dilating tip.

6. The hemostatic stabilization system of claim 1, wherein the hemostatic port comprises a single working channel.

7. The hemostatic stabilization system of claim 1, wherein the hemostatic port comprises more than one working channel.

8. A hemostatic stabilization system for hemostatically accessing a bodily organ, comprising:
a hemostatic cup comprising a proximal end, a distal end, and a wall extending therebetween, the hemostatic cup further comprising a tissue attachment edge at the distal end, and a sealing surface at the proximal end; and
a hemostatic valve attached to the sealing surface, the hemostatic cup defining a working channel configured to receive an instrument to be inserted into the bodily organ,
wherein the hemostatic cup defines a vacuum chamber configured to adhere the hemostatic cup to the bodily organ when a vacuum source is coupled thereto and when the tissue attachment edge is brought into contact with the bodily organ, and
wherein the hemostatic cup is configured to maintain a hemostatic environment when the instrument is inserted into the bodily organ through the hemostatic valve.

9. The hemostatic stabilization system of claim 8, wherein the hemostatic cup comprises a frustoconical shape.

10. The hemostatic stabilization system of claim 8, wherein the hemostatic cup further comprises a port channel through which the hemostatic port is provided.

11. The hemostatic stabilization system of claim 8, further comprising a second vacuum chamber.

12. The hemostatic stabilization system of claim 8, wherein the hemostatic port comprises an incising, self-dilating tip.

13. The hemostatic stabilization system of claim 8, wherein the hemostatic port comprises a single working channel.

14. The hemostatic stabilization system of claim 8, wherein the hemostatic port comprises more than one working channel.

15. A method of hemostatically accessing a patient's ventricle, comprising:
placing a tissue attachment edge of a hemostatic stabilization system to tissue surrounding the apex of the patient's heart;
applying suction to a vacuum port of the hemostatic stabilization system to secure the hemostatic stabilization system to the patient's heart;
inserting a hemostatic port through the tissue surrounded by the hemostatic stabilization system and into the patient's ventricle; and
performing a procedure within the patient's heart with an instrument inserted into the ventricle via the hemostatic port.

16. The method of claim 15, further comprising penetrating the tissue surrounded by the hemostatic stabilization system with an incising, self-dilating tip of the hemostatic port.

17. The method of claim 15, wherein the procedure comprises performing a valvuloplasty on a diseased valve within the patient's heart.

18. The method of claim 15, wherein the procedure comprises removing the diseased tissue of a valve within the patient's heart.

19. The method of claim 15, wherein the procedure comprises delivering a replacement valve to the patient's heart.

20. The method of claim 15, further comprising closing an opening formed in the tissue surrounded by the hemostatic stabilization system.

21. A method of hemostatically accessing a patient's ventricle, comprising:
providing a hemostatic stabilization system, comprising a tissue attachment edge at the hemostatic stabilization system's distal end, a sealing surface at the hemostatic stabilization system's proximal end, and a wall extending therebetween, the hemostatic stabilization system further comprising a vacuum port in communication with a vacuum chamber defined in part by the wall and a hemostatic valve positioned at the sealing surface;
placing the tissue attachment edge in contact with tissue surrounding the apex of the patient's heart;
applying suction to the vacuum port to secure the hemostatic stabilization system to the patient's heart;
inserting an instrument through the hemostatic valve into the patient's ventricle; and
performing a procedure within the patient's heart with an instrument inserted into the ventricle via the hemostatic valve.

22. The method of claim 21, further comprising penetrating the tissue surrounded by the hemostatic stabilization system with an incising, self-dilating tip of the hemostatic port.

23. The method of claim 21, wherein the procedure comprises performing a valvuloplasty on a diseased valve within the patient's heart.

24. The method of claim 21, wherein the procedure comprises removing the diseased tissue of a valve within the patient's heart.

25. The method of claim 21, wherein the procedure comprises delivering a replacement valve to the patient's heart.

26. The method of claim 21, further comprising closing an opening formed in the tissue surrounded by the hemostatic stabilization system.
